(19) United States
(12) Patent Application Publication
(10) Pub. No.: US 2010/0043084 A1
(43) Pub. Date: Feb. 18, 2010

(54) Cotton varieties having a main stem load structure

(76) Inventors: James M. Olvey, Tempe, AZ (US); James Michael Olvey, II, Gilbert, AZ (US)

Correspondence Address: JAMES M. OLVEY, P.O. Box 1440, Maricopa, AZ, 85239, US

(21) Appl. No.: 12/462,873

(22) Filed: Aug. 11, 2009

Related U.S. Application Data
(60) Provisional application No. 61/088,636, filed on Aug. 13, 2008.

(51) Int. Cl. A01H 5/00; A01H 1/02; A01H 5/10; C07H 21/00; C12N 5/04

(52) U.S. Cl. 800/260; 800/314; 800/301; 536/23.1; 435/410

(57) ABSTRACT

The present invention relates to a cotton plant, seed, variety and hybrid. More specifically, the invention relates to a cotton plant having a mutant allele designated OA1 which results in a cotton plant with a Main Stem Load structure when present in either the heterozygous or homozygous state. The invention also relates to crossing cotton plants containing the OA1 mutant allele with cotton plants lacking the OA1 mutant allele to produce novel types of cotton plants.

### BACKGROUND OF THE INVENTION

The present invention relates to a mutant allele of cotton designated OA1, which results in a Main Stem Load structure in a cotton plant. The present invention also relates to a cotton seed, a cotton plant and parts of a cotton plant and a cotton hybrid plant, having the OA1 mutant allele. In addition, the present invention is directed to transferring the OA1 mutant allele in the cotton plant to other cotton varieties and species. All publications cited in this application are herein incorporated by reference.

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single cultivar an improved combination of desirable traits from the parental germplasm. In cotton, the important traits include higher fiber (lint) yield, earlier maturity, improved fiber quality, resistance to diseases and insects, resistance to drought and heat, and improved agronomic traits.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination and the number of hybrid offspring from each successful cross. Forward Crossing refers to any system of inbred line development, irrespective of the number of loci involved or the balance of favorable alleles among the parents of the population, involving the creation of a source population followed by inbreeding with selection, with the goal of recovering an improved line for one or more traits (e.g., pedigree selection).

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to popular cultivars in environments representative of the commercial target area(s) for three or more years. The best lines having superiority over the popular cultivars are candidates to become new commercial cultivars. Those lines still deficient in a few traits are discarded or utilized as parents to produce new populations for further selection.

These processes, which lead to the final step of marketing and distribution are usually from seven to twelve years from the time the first cross is made. Therefore, development of new cultivars is a time-consuming process that requires precise forward planning, efficient use of resources, and a minimum of changes in direction.

A most difficult task is the identification of individual cultivars that are genetically superior because, for most traits the true genotypic value is masked by other confounding plant traits or environmental factors. One method of identifying a superior plant is to observe its performance relative to other experimental lines and widely grown standard cultivars. For many traits a single observation is inconclusive, and replicated observations over time and space are required to provide a good estimate of a line's genetic worth.

The goal of a commercial cotton breeding program is to develop new, unique and superior cotton cultivars. The breeder initially selects and crosses two or more parental lines, followed by generation advancement and selection, thus producing many new genetic combinations. The breeder can theoretically generate billions of different genetic combinations via this process. The breeder has no direct control over which genetic combinations will arise in the limited population size which is grown. Therefore, two breeders will never develop the same line having the same traits.

Each year, the plant breeder selects the germplasm to advance to the next generation. This germplasm is grown under unique and different geographical, climatic and soil conditions, and further selections are then made, during and at the end of the growing season. The lines which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce, with any reasonable likelihood, the same cultivar twice by using the exact same original parents and the same selection techniques. This unpredictability results in the expenditure of large amounts of research monies to develop superior new cotton cultivars.

Pureline cultivars of cotton are commonly bred by hybridization of two or more parents followed by selection. The complexity of inheritance, the breeding objectives and the available resources influence the breeding method. Pedigree breeding, recurrent selection breeding and backcross breeding are breeding methods commonly used in self-pollinated crops such as cotton. These methods refer to the manner in which breeding pools or populations are made in order to combine desirable traits from two or more cultivars or various broad-based sources. The procedures commonly used for selection of desirable individuals or populations of individuals are called mass selection, plant-to-row selection, and single seed descent or modified single seed descent. One, or a combination of these selection methods, can be used in the development of a cultivar from a breeding population.

Pedigree breeding is primarily used to combine favorable genes into a totally new cultivar that is different in many traits than either parent used in the original cross. It is commonly used for the improvement of self-pollinating crops. Two parents which possess favorable, complementary traits are crossed to produce an F₁ (filial generation 1). An F₂ population is produced by selfing F₁ plants. Selection of desirable individual plants may begin as early as the F₂ generation wherein maximum gene segregation occurs. Individual plant selection can occur for one or more generations. Successively, seed from each selected plant can be planted in individual identified rows or hills, known as progeny rows or progeny hills, to evaluate the line and to increase the seed quantity or to further select individual plants. Once a progeny row or progeny hill is selected as having desirable traits it becomes what is known as a breeding line that is specifically identifiable from other breeding lines that were derived from the same original population. At an advanced generation (i.e., F₅ or higher) seed of individual lines are evaluated in replicated testing. At an advanced stage the best lines or a mixture of phenotypically similar lines from the same original cross are tested for potential release as new cultivars.

The single seed descent procedure, in the strict sense, refers to planting a segregating population, harvesting one seed from every plant, and combining these seeds into a bulk which is planted in the next generation. When the population has been advanced to the desired level of inbreeding, the plants from which lines are derived will each trace to different F₂ individuals. Primary advantages of the seed descent procedures are to delay selection until a high level of homozygosity (e.g., lack of gene segregation) is achieved in individual plants, and to move through these early generations quickly, usually through using winter nurseries.

The modified single seed descent procedures involve harvesting multiple seed (i.e., a single lock or a simple boll) from each plant in a population and combining them to form a bulk. Part of the bulk is used to plant the next generation and part is put in reserve. This procedure has been used to save labor at harvest and to maintain adequate seed quantities of the population.

Selection for desirable traits can occur at any segregating generation (F₂ and above). Selection pressure is exerted on a population by growing the population in an environment where the desired trait is maximally expressed and the individuals or lines possessing the trait can be identified. For instance, selection can occur for disease resistance when the plants or lines are grown in natural or artificially-induced disease environments, and the breeder selects only those individuals having little or no disease and are thus assumed to be resistant.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Isozyme Electrophoresis and RFLPs have been widely used to determine genetic composition. Shoemaker and Olsen, (Molecular Linkage Map of Soybean (Glycine max L. Merr.) p 6.131-6.138 in S. J. O'Brien (ed) Genetic Maps: Locus Maps of Complex Genomes, Cold Spring Harbor Laboratory Press, Cold Spring Harbor, N.Y., (1993)) developed a molecular genetic linkage map that consisted of 25 linkage groups with about 365 RFLP, 11 RAPD and three classical markers and four isozyme loci. See also, Shoemaker, R. C., RFLP Map of Soybean, p 299-309, in Phillips, R. L. and Vasil, I. K. (eds.) DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225, 1997.) SNPs may also be used to identify the unique genetic composition of the invention and progeny varieties retaining that unique genetic composition. Various molecular marker techniques may be used in combination to enhance overall resolution.

Molecular markers, which includes markers identified through the use of techniques such as Isozyme Electrophoresis, RFLPs, RAPDs, AP-PCR, DAF, SCARs, AFLPs, SSRs, and SNPS, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. For example, molecular markers are used in cotton breeding for selection of desirable traits. See Preetha, S., et al. Biotechnology and Molecular Biology Review. 3(2): 3245, April 2008. The markers can also be used to select toward the genome of the recurrent parent and against the markers of the donor parent. Using this procedure can attempt to minimize the amount of genome from the donor parent that remains in the selected plants. It can also be used to reduce the number of crosses back to the recurrent parent needed in a backcrossing program. The use of molecular markers in the selection process is often called Genetic Marker Enhanced Selection. Molecular markers may also be used to identify and exclude certain sources of germplasm as parental varieties or ancestors of a plant by providing a means of tracking genetic profiles through crosses as discussed more fully hereinafter.

Mutation breeding is another method of introducing new traits into cotton varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogues like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

Descriptions of other breeding methods that are commonly used for different traits and crops can be found in one of several reference books (e.g., Allard, 1960; Simmonds, 1979; Sneep, et al. 1979; Fehr, 1987).

Proper testing should detect any major faults and establish the level of superiority or improvement over current cultivars. In addition to showing superior performance, there must be a demand for a new cultivar that is compatible with industry standards or which creates a new market. The introduction of a new cultivar will incur additional costs to the seed producer, the grower, processor and consumer; may require special advertising, marketing, and commercial production practices, as well as new product utilization. The testing that precedes the release of a new cultivar should take into consideration research and development costs as well as technical superiority of the final cultivar. For seed-propagated cultivars, it must be feasible to produce seed easily and economically.

Cotton, including Gossypium hirsutum (Upland/Acala) and Gossypium barbadense (Pima), is an important and valuable field crop. Thus, a continuing goal of cotton plant breeders is to develop stable, high-yielding cotton cultivars of both cotton species that are agronomically sound. The reasons for this goal are obviously to maximize the amount and quality of the fiber produced on the land used and to supply fiber, oil and food for animals and humans. To accomplish this goal, the cotton breeder must select and develop plants that have the traits that result in superior cultivars.

The development of new cotton cultivars requires the evaluation and selection of parents and the crossing of these parents. The lack of predictable success of a given cross requires that a breeder, in any given year, make several crosses with the same or different breeding objectives.

The cotton flower is monoecious in that the male and female structures are in the same flower. The crossed or hybrid seed is produced by manual crosses between selected parents. Floral buds of the parent that is to be the female are emasculated prior to the opening of the flower by manual removal of the male anthers. At flowering, the pollen from flowers of the parent plants designated as male, are manually placed on the stigma of the previous emasculated flower. Seed developed from the cross is known as first generation (F₁) hybrid seed. Planting of this seed produces F₁ hybrid plants of which half their genetic component is from the female parent and half from the male parent. Segregation of genes begins at meiosis thus producing second generation (F₂) seed. Assuming multiple genetic differences between the original parents, each F₂ seed has a unique combination of genes.

The foregoing examples of the related art and limitations related therewith are intended to be illustrative and not exclusive. Other limitations of the related art will become apparent to those of skill in the art upon a reading of the specification.

### BRIEF DESCRIPTION OF THE FIGURES

FIG. 1 shows an example of a cotton plant structure containing the OA1 mutant allele. Open circles represent bolls and closed circles represent leaves.

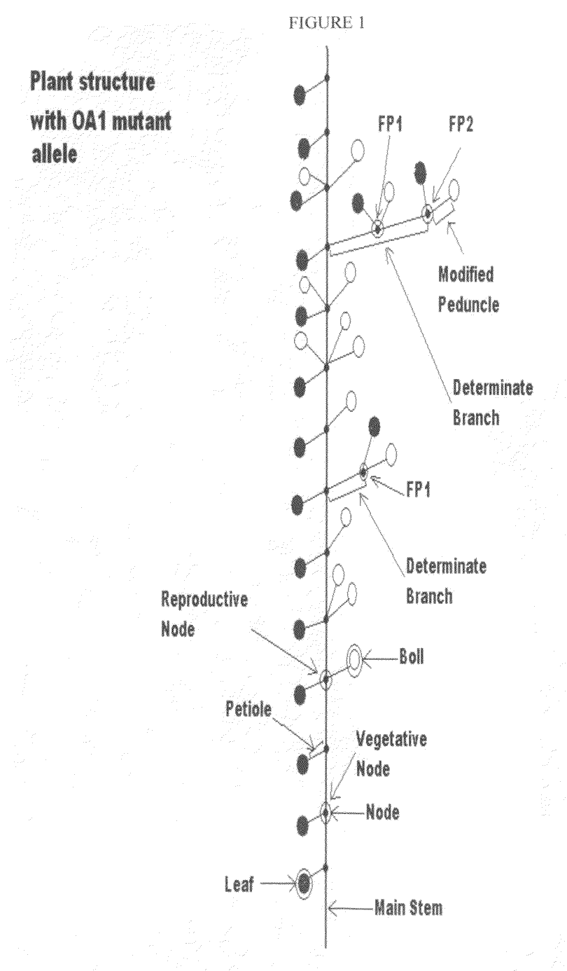

FIG. 2 shows an example of a cotton plant structure that does not contain the OA1 mutant allele. Open circles represent bolls and closed circles represent leaves.

### SUMMARY OF THE INVENTION

The following embodiments and aspects thereof are described in conjunction with systems, tools, and methods which are meant to be exemplary and illustrative, not limiting in scope. In various embodiments, one or more of the above-described problems have been reduced or eliminated, while other embodiments are directed to other improvements.

The present invention provides a mutant allele designated OA1 derived from cotton that is phenotypically expressed by an unexpected Main Stem Load structure when present in either the heterozygous or homozygous state. This mutant allele has been determined to be a dominant or partially dominant gene. The invention further provides plants, seeds, and other plant parts such as pollen and ovules containing the OA1 mutant allele.

The invention also provides methods for introducing the OA1 mutant allele into plants by crossing a plant which lacks the OA1 mutant allele with a plant that has the OA1 mutant allele, selfing the resulting generations and then selecting the plants exhibiting a Main Stem Load structure containing the OA1 mutant allele.

In another aspect, the present invention provides a method for producing a hybrid cotton seed comprised of crossing a first parent plant with a second parent plant and harvesting the resultant hybrid cotton seed, wherein either one or both parents contain the OA1 mutant allele. The hybrid cotton seeds, plant and parts thereof produced by such method are also part of the invention.

In another aspect, the present invention relates to any cotton plant or seed having the OA1 mutant allele.

In another aspect, the present invention relates to any hybrid cotton plant or seed having the OA1 mutant allele.

In another aspect, the present invention provides for a cotton plant having a reduced number of branches and leaves.

In another aspect, the present invention provides for a cotton plant having a greater number of bolls off of the main stem.

In another aspect, the present invention provides regenerable cells for use in tissue culture of cotton plants having the OA1 mutant allele. The tissue culture will preferably be capable of regenerating plants having the physiological and morphological characteristics of the foregoing cotton plant, and of regenerating plants having substantially the same genotype as the foregoing cotton plant. Preferably, the regenerable cells in such tissue cultures will be embryos, protoplasts, meristematic cells, callus, pollen, leaves, anthers, pistils, roots, root tips, flowers, seeds, or stems. Still further, the present invention provides cotton plants regenerated from the tissue cultures of the invention.

In addition to the exemplary aspects and embodiments described above, further aspects and embodiments will become apparent by study of the following descriptions.

### DEFINITIONS

In the description and tables which follow, a number of terms are used. In order to provide a clear and consistent understanding of the specification and claims, including the scope to be given such terms, the following definitions are provided:

Allele. Allele is any of one or more alternative forms of a gene, all of which relate to one trait or characteristic. In a diploid cell or organism, the two alleles of a given gene occupy corresponding loci on a pair of homologous chromosomes.

Area. The ratio of accumulated areas of all the trash particles to the area of the viewing of the instrument.

B. As used herein, “B” is the part of the Hunter's Scale that indicates yellowness; cotton ranges from 4 to 18 on the scale.

Backcrossing. Backcrossing is a process in which a breeder repeatedly crosses hybrid progeny back to one of the parents, for example, a first generation hybrid F. with one of the parental genotypes of the F. hybrid.

Boll. The flowering and/or fruiting structure of a cotton plant which matures into a flower or a boll.

Branch. An auxiliary (lateral) shoot off the main stem of the plant.

Branch Length to Boll (BRB). As used herein, the term “Branch length to boll” means the average distance of a boll from the main stem in centimeters.

Cell. Cell as used herein includes a plant cell, whether isolated, in tissue culture or incorporated in a plant or plant part.

Classers Leaf (CLLF). This is similar to LF except that it refers to a table from the USDA that is the latest adjusted values for this reading as below:

| Class of LF | % Area | |
|---|---|---|
| 1 | 0.1 | <.19 |
| 2 | 0.2 | .20-.29 |
| 3 | 0.3 | .30-.39 |
| 4 | 0.4 & 0.5 | .40-.49 |
| 5 | 0.6 & 0.7 | .50-.59 |
| 6 | 0.8 > 1.0 | .80-1.09 |
| 7 | 1.1 > 1.4 | 1.10-1.49 |
| BG | >1.5 | >1.5 |

Colgrade (CG). Means the intersection of the B and Rd on the Universal Standard for Grade of American Upland Cotton chart. The Higher the number, the more yellowness is observed in the cotton fibers.

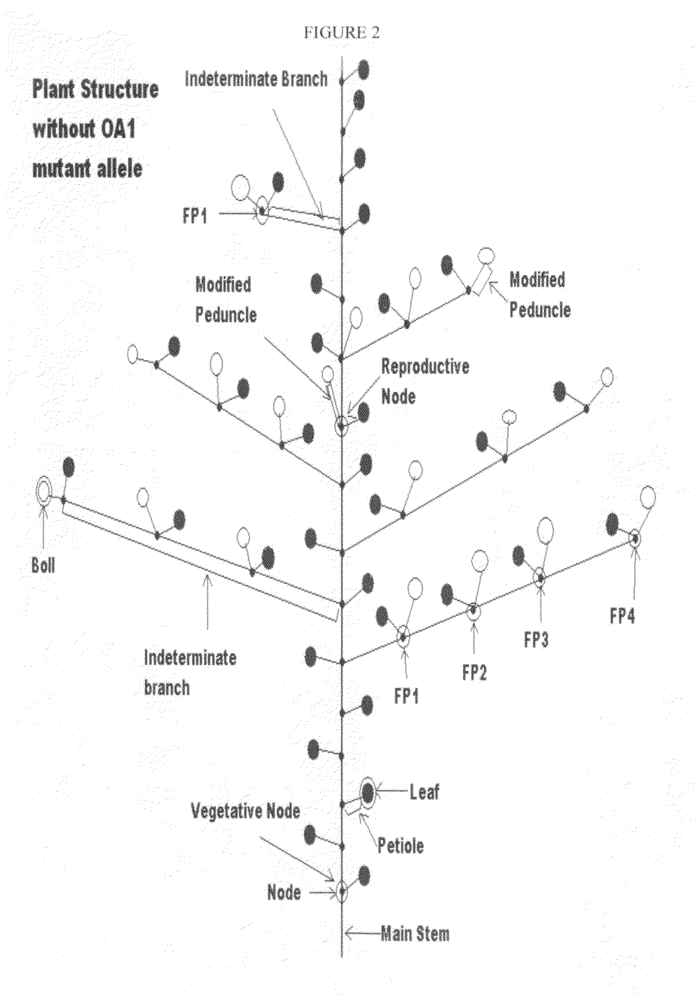

Conventional cotton plant. As used herein, the term “conventional cotton plant” is defined as a cotton plant having indeterminate branches. Conventional cotton plants do not contain the OA1 mutant allele. An example of a conventional cotton plant is illustrated in FIG. 2.

Count Strength Product (CSP). In determining yarn strength, “count strength product” is the product of the English yarn number and the skein break value.

Determinate Branch. As used herein, the term “determinate branch” is defined as a branch off the main stem whose length is limited. In this application, a determinate branch means a branch that terminates after the first or second fruiting position. A determinate branch is generally shorter than an indeterminate branch because there are less fruiting positions on a determinate branch.

Disease Resistance. As used herein, the term “disease resistance” is defined as the ability of plants to restrict the activities of a specified pest, such as an insect, fungus, virus, or bacterial.

Disease Tolerance. As used herein, the term “disease tolerance” is defined as the ability of plants to endure a specified pest (such as an insect, fungus, virus or bacteria) or an adverse environmental condition and still perform and produce in spite of this disorder.

Essentially all of the physiological and morphological characteristics. A plant having essentially all of the physiological and morphological characteristics of the recurrent parent, except for the characteristics derived from the converted trait.

Fiber Elongation (E1). As used herein, the term “fiber elongation” is defined as the measure of elasticity of a bundle of fibers as measured by High Volume Instrumentation (HVI).

Fiber Length (Len). As used herein, the term “fiber length” is defined as the longest 2.5% span length in inches of fiber as measured by HVI.

Fiber Strength (T1). As used herein, the term “fiber strength” is defined as the force required to break a bundle of fibers as measured in grams per millitex on the HVI.

Forward Crossing. As used herein, the term “forward crossing” is defined as any system of inbred line development, irrespective of the number of loci involved or the balance of favorable alleles among the parents of the population, with the goal of recovering an improved line for one or more traits (i.e., pedigree selection).

FP1 part of the branch. The first fruiting position closest to but not on the main stem on a branch that makes a prophyll or a leaf and terminates in a flower or a boll.

FP2 part of the branch. The second fruiting position closest to but not on the main stem on a branch that makes a prophyll or a leaf and terminates in a flower or a boll.

FP3 part of the branch. The third fruiting position closest to but not on the main stem on a branch that makes a prophyll or a leaf and terminates in a flower or a boll.

FP4 part of the branch. The fourth fruiting position closest to but not on the main stem on a branch that makes a prophyll or a leaf and terminates in a flower or a boll.

FP5 part of the branch. The fifth fruiting position closest to but not on the main stem on a branch that makes a prophyll or a leaf and terminates in a flower or a boll.

Fruiting Branches (FB). As used herein, the term “fruiting branches” is defined as a branch off of the main stem which has at least one node and one boll per plant.

Fruiting Positions on the Branches (FTP). As used herein, the term “fruiting positions on the branches” is defined as an average number of the total fruiting positions on determinate and/or indeterminate branches per plant

Fruiting Positions off of the Main Stem (FTP off MS). As used herein, the term “fruiting positions off of the main stem” is defined as an average of the total number of fruiting positions on the main stem per plant.

Fruiting Position (FP). As used herein, the term “fruiting position” is a node that has at least one petiole and/or at least one modified peduncle, which has resulted in a flower or a boll along a determinate branch, indeterminate branch or main stem. A fruiting position is always a node but a node is not always a fruiting position.

Fusarium Race-4. Fusarium oxysporum f. sp. Vasinfectum is a soilbourne fungus that causes vascular wilt in cotton.

Genome. Genome refers to the total hereditary makeup of an organism.

Genotype. Genotype refers to the genetic constitution of a cell or organism.

Germplasm. Germplasm means plant genetic materials that serve as a reservoir of genes for cultivar improvement or for research. Germplasm includes all types of plants such as inbreds, hybrids, varieties, F2, F3, F4 through F12 generations.

Gin Turnout. As used herein, the term “gin turnout” is defined as a fraction of lint in a machine harvested sample of seed cotton (lint, seed, and trash).

Ginning. Ginning refers to the process of removing seeds from the long, commercially viable, fibers with a cotton gin.

Height (Ht). As used herein, the term “height” is defined as the average height in centimeters, as measured from the ground level to the top or apex of the plant.

Indeterminate branch. As used herein, the term “indeterminate branch” is defined as a branch whose fruiting positions could, in theory, go to infinity. The number of fruiting positions on an indeterminate branch can vary. An indeterminate branch is generally longer than a determinate branch.

Lint/boll. As used herein, the term “lint/boll” is the weight of lint percentage of seed cotton per boll.

Lint Index. As used herein, the term “lint index” refers to the weight of lint per seed in milligrams per 100 seeds.

Lint Percent. As used herein, the term “lint percent” is defined as the lint (fiber) fraction of a hand-picked sample of seed cotton (lint and seed).

Lint Weight. As used herein, the term “lint weight” is defined as the weight of long or commercially viable fibers from cotton seed.

LF. The trash content in the cotton fiber; this is a code which is assigned to a sample based on where the sample falls according to the trash levels determined during calibration.

Locus. A locus refers to the chromosomal location or DNA sequence of a trait such as, for example, male sterility, herbicide tolerance, insect resistance, disease resistance, or improved yield. The trait may be, for example, conferred by a naturally occurring gene introduced into the genome of the variety by backcrossing, a natural or induced mutation, or a transgene introduced through genetic transformation techniques. The same loci on homologous chromosomes of a tetraploid, such as cotton, may comprise one (homozygous) or two (heterozygous) alleles.

Main Stem. As used herein, the term “main stem” refers to the primary vertical stem of the plant that supports branches, leaves, flowers or fruit; there is only one main stem per plant.

Main Stem Load (MSL). As used herein, the term “Main Stem Load” is defined as a cotton plant that has no indeterminate branches. Most bolls are located off the main stem from the modified peduncle. The other bolls are located off a determinate branch from the modified peduncle. The Main Stem Load is the phenotypic expression of the OA1 mutant allele.

Maturity Rating (MR). As used herein, the term “maturity rating” is defined as an HVI rating on the amount of bolls on the plant.

Micronaire (Mic). As used herein, the term “micronaire” is defined as a measure of the fineness of the fiber. Within a cotton cultivar, micronaire is also a measure of maturity. Micronaire differences are governed by changes in perimeter or in cell wall thickness, or by changes in both. Within a cultivar, cotton perimeter is fairly constant and maturity will cause a change in micronaire. Consequently, micronaire has a high correlation with maturity within a variety of cotton. Maturity is the degree of development of cell wall thickness. Micronaire may not have a good correlation with maturity between varieties of cotton having different fiber perimeter. Micronaire values range from about 2.0 to 6.0:

| | | |
|---|---|---|
| Below 2.9 | Very | Possible small perimeter but mature (good fiber), |
| | fine | or large perimeter but immature (bad fiber). |
| 2.9 to 3.7 | Fine | Various degrees of maturity and/or perimeter. |
| 3.8 to 4.6 | Average | Average degree of maturity and/or perimeter. |
| 4.7 to 5.5 | Coarse | Usually fully developed (mature), but larger |
| | | perimeter. |
| 5.6+ | Very | Fully developed, large-perimeter fiber. |
| | coarse | |

Modified Peduncle. As used herein, the term “peduncle” means the stalk by which a flower or a boll is attached.

Naturally colored cotton. As used herein, the term “naturally colored cotton” means any species of cotton with a lint color other than white (i.e. green, brown, red and the like).

Node. As used herein, the term “node” refers to the point on a plant where a petiole and possibly one or more peduncles protrude.

Number of back to back bolls. As used herein, the term “number of back to back bolls” is defined as the average number of modified peduncles which produce two bolls arising from the same node.

Number of bolls off of the main stem (B off MS). As used herein, the term “number of bolls off of the main stem” means the average number of nodes located on the main stem of the plant that makes a prophyll and/or a leaf and terminate in a flower which produces a boll.

Number of Nodes (#nodes). As used herein, the “number of nodes” means the average total number of nodes on the plant.

Number of FP1 (#FP1). As used herein, means the average number of bolls off the FP1 part of the branch for a given plant.

Number of FP2 (#FP2). As used herein, means the average number of bolls off the FP2 part of the branch for a given plant.

Number of FP3 (#FP3). As used herein, means the average number of bolls off the FP3 part of the branch for a given plant.

Number of FP4 (#FP4). As used herein, means the average number of bolls off the FP4 part of the branch for a given plant.

Number of FP5 (#FP5). As used herein, means the average number of bolls off the FP5 part of the branch for a given plant.

Petiole. As used herein, the term “petiole” refers to the stalk by which a leaf is attached.

Pima cotton. As used herein, the term “pima cotton” means any plant of the species Gossypium barbadense. G. barbadense is used interchangeably with pima cotton and is meant herein to mean pima cotton and vice versa.

Plant. As used herein, the term “plant” includes reference to an immature or mature whole cotton plant, including a plant from which seed or fibers or anthers have been removed. A seed or embryo that will produce the plant is also considered to be the plant. Plant also includes plant cells, plant protoplasts, plant cell tissue cultures from which cotton plants can be regenerated, plant calli, plant clumps, and plant cells that are intact in plants or parts of plants, such as embryos, pollen, ovules, flowers, leaves, roots, root tips, anthers, and the like.

Plant parts. As used herein, the term “plant parts” (or a cotton plant, or a part thereof) includes protoplasts, leaves, stems, roots, root tips, anthers, seed, fiber, boll, embryo, pollen, ovules, cotyledon, hypocotyl, flower, shoot, tissue, petiole, cells, meristematic cells and the like.

Prophyll. As used herein, the term “prophyll” refers to a plant structure that resembles a leaf.

Quantitative trait loci (QTL). Quantitative trait loci (QTL) refer to genetic loci that control to some degree numerically representable traits that are usually continuously distributed.

RD. The unit of measure for reflectance. Higher RD values indicate higher grades of cotton; cotton ranges from 40 to 85 RD.

Reduced number of branches and leaves. As used herein, means a decreased number of branches and leaves per plant as compared to conventional cotton plants.

Regeneration. Regeneration refers to the development of a plant from tissue culture or vegetative cuttings.

Reproductive node (RN). As used herein, the term “reproductive node” means a slightly enlarged portion of a stem where leaves and buds arise and where branches can originate; the first leaf and protective scale of a branch or modified peduncle.

Seed/boll. As used herein, the term “seed/boll” refers to the number of seeds per boll.

Seedcotton. As used herein, the term “seedcotton” refers to the weight of both lint and seed.

Seedcotton/boll. As used herein, the term “seedcotton/boll” refers to the weight of seedcotton (lint and seed) per boll.

Seed Weight (Seed Wt). As used herein, the term “seed weight” is the weight of 100 seeds in grams.

Short Fiber Index (SFI). This is an estimate of the short fiber content in the long cotton fiber sample.

Single trait Converted (Conversion). Single trait converted (conversion) plant refers to plants which are developed by a plant breeding technique called backcrossing or via genetic engineering wherein essentially all of the desired morphological and physiological characteristics of a variety are recovered in addition to the single trait transferred into the variety via the backcrossing technique or via genetic engineering.

Spinning Consistency Index (SCI). This is a calculated value based on a regression equation that takes into account all HVI properties.

Total Bolls (TB). As used herein, the term “total bolls” means the total number of bolls on a cotton plant. ((the sum of FP1 through FP5)+B off MS+Surplus). Since the five feet worth of plants were selected out of the field and analyzed at a different location, occasionally there would be a boll or two break off from a plant and those few comprise “surplus” which are added to the total number of bolls on the plant.

Uniformity Ratio (UR). As used herein, the term “uniformity ratio” is defined as a measure of the relative length uniformity of a bundle of fibers as measured by HVI.

Upland/Acala cotton. “Upland/Acala cotton” means any plant of the species Gossypium hirsutum. G. hirsutum is used interchangeably with upland/acala cotton and is meant herein to mean upland/acala cotton and vice versa.

Vegetative Nodes. As used herein, the term “vegetative nodes” is defined as the number of nodes from the cotyledonary node to the first fruiting branch or modified peduncle on the main stem of the plant.

### DETAILED DESCRIPTION OF THE INVENTION

This application claims benefit of my provisional application no. 61088636 filed Aug. 13, 2008 with additional corrections. The present invention relates to a mutant allele designated OA1 in the genus Gossypium that results in cotton varieties with a Main Stem Load structure reducing the number of branches and leaves when compared to conventional or commercial cotton plants that do not contain the OA1 mutant allele. The present invention allows for cotton plants having greater harvest efficiencies, an earlier maturity, a higher percentage of boll retention, higher plant population densities and less vegetation (i.e., leaves). The advantages of developing a cotton plant with reduced number of branches and leaves are multiple and substantial, the greatest of which are reduced cost (economic) and greater production and harvesting efficiencies (environmental).

The present invention relates to a mutant allele designated OA1 and was discovered from a selection derived from a proprietary cross between M-VCL and 86-106 which resulted in a cotton plant having a Main Stem Load structure. Cotton seed and plant as used herein means any cotton cultivars (varieties) of G. hirsutum, G. barbadense or hybrids of G. hirsutum, hybrids of G. barbadense or any interspecific hybrid of G. hirsutum×G. barbadense. The present invention also relates to a cotton seed, a cotton plant and plant parts which comprise the OA1 mutant allele.

The OA1 mutant allele of the present invention can be introgressed into any cotton cultivar or hybrid. A plant of the present invention can be obtained by crossing a plant either heterozygous or homozygous for the claimed OA1 mutant allele with any cotton cultivar or cotton hybrid not containing the OA1 mutant allele. The plant containing the OA1 mutant allele can be any cotton cultivar including a cultivar in which the OA1 mutant allele has been previously genetically fixed. The OA1 mutant allele may then be transmitted by sexual crossing to other cotton cultivars or cotton hybrids if desired.

The OA1 mutant allele will advantageously be introduced into cotton cultivars that contain other desirable genetic traits such as resistance to disease or other pests, drought tolerance, increased yield, naturally colored cotton, and the like.

The invention further provides methods for developing cotton plants having the OA1 mutant allele in a plant breeding program using plant breeding techniques including but not limited to, parental selection and hybrid development, recurrent selection, forward crossing, backcrossing, pedigree breeding, restriction fragment length polymorphism enhanced selection, genetic marker enhanced selection and transformation. Seeds, cotton plants, and parts thereof produced by such breeding methods are also part of the invention.

This invention is also directed to methods for producing a cotton plant by crossing a first parent cotton plant with a second parent cotton plant, wherein the first or second cotton plant is a cotton plant containing the OA1 mutant allele. Further, both the first and second parent cotton plants may contain the OA1 mutant allele (e.g., self-pollination). Therefore, any methods using a cotton plant with the OA1 mutant allele are part of this invention including but not limited to: selfing, forward crossing, backcrosses, hybrid breeding, and crosses to populations. Any plants produced using cotton plants having the OA1 mutant allele as a parent are within the scope of this invention. As used herein, the term “plant” includes plant cells, plant protoplasts, plant cells of tissue culture from which cotton plants can be regenerated, plant calli, plant clumps, and plant cells that are intact in plants or parts of plants, such as pollen, flowers, embryos, ovules, bolls, seeds, leaves, stems, roots, anthers, pistils and the like.

The present invention also encompasses a cotton plant having no branches off of the main stem.

The present invention also encompasses a cotton plant having an average of three or fewer determinate branches.

The present invention encompasses a cotton plant which produces at least five bolls off of the main stem.

The present invention also encompasses a cotton plant having no bolls off of the FP1 part of any branch.

The present invention also encompasses a cotton plant having less than two fruiting positions on the determinate branches.

Another important aspect of the present invention is an earlier maturity due to the production of less vegetation (i.e., fewer branches and leaves).

Another important aspect of the present invention is that it provides for greater harvest efficiencies. The greater harvest efficiency provides for a substantial two-fold benefit: due to the form of the plant (upright, none to few branches and reduced number of leaves) the grower can plant cotton plants in a higher density, which allows more bolls to be harvested per unit area.

Another important aspect of the present invention is that it provides for a higher percentage of boll retention in that bolls that are produced off of the main stem are more likely to be retained when compared to bolls that are not produced off of the main stem.

The present invention also encompasses a cotton plant regenerated from a tissue culture of a cotton cultivar or cotton hybrid plant having the OA1 mutant allele. As is well known in the art, tissue culture of cotton can be used for the in vitro regeneration of a cotton plant. Tissue culture of various tissues of cotton and regeneration of plants therefrom is well known and widely published.

### EXAMPLES

The following examples further describe the materials and methods used in carrying out the invention and the subsequent results. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

### Discovery/Development of the Original Plant Containing the OA1 Mutant Allele

The OA1 mutant allele was discovered from a selection derived from a proprietary cross between M-VCL and 86-106, which unexpectedly resulted in a cotton plant having a Main Stem Load structure.

### Example 2

### Introgression of the OA1 Mutant Allele into Upland/Acala Cotton Plants Lacking the OA1 Mutant Allele

Cotton plants containing the OA1 mutant allele of the present invention are crossed with different upland/acala cotton plants lacking the OA1 mutant allele. The progeny seeds are sown and grown under controlled greenhouse/field conditions. Desirable plants are selected and self-pollinated or crossed again with chosen breeding lines. Cotton plants containing the OA1 mutant allele are derived from these crosses either in the first generation population or the subsequent generation populations selecting for the Main Stem Load structure. The main characteristics in the selection process are (1) no branches, (2) reduction in the number of leaves, (3) Upland/Acala Characteristics, (4) uniqueness, (5) fiber characteristics, (6) disease resistance, resulting in a commercially viable cotton variety.

### Example 3

### Introgression of the OA1 Mutant Allele into Pima Cotton Plants Lacking the OA1 Mutant Allele

Cotton plants containing the OA1 mutant allele of the present invention are crossed with different pima cotton plants lacking the OA1 mutant allele. The progeny seeds are sown and grown under controlled greenhouse/field conditions. Desirable plants are selected and self-pollinated or crossed again with chosen breeding lines. Cotton plants containing the OA1 mutant allele are derived from these crosses either in the first generation population or the subsequent generation populations selecting for the Main Stem Load structure. The main characteristics in the selection process are (1) no branches, (2) reduction in the number of leaves, (3) Pima Characteristics, (4) uniqueness, (5) fiber characteristics, (6) disease resistance, resulting in a commercially viable cotton variety.

### Example 4

### Introgression of the OA1 Mutant Allele into Naturally Colored Cotton Varieties Lacking the OA1 Mutant Allele

Cotton plants containing the OA1 mutant allele of the present invention are crossed with different naturally colored cotton plants lacking the OA1 mutant allele. The progeny seeds are sown and grown under controlled greenhouse/field conditions. Desirable plants are selected and self-pollinated or crossed again with chosen breeding lines. Cotton plants containing the OA1 mutant allele are derived from these crosses either in the first generation population or the subsequent generation populations selecting for the Main Stem Load structure. The main characteristics in the selection process are (1) no branches, (2) reduction in the number of leaves, (3) Naturally colored cotton characteristics, (4) uniqueness, (5) fiber characteristics, (6) disease resistance, resulting in a commercially viable cotton variety.

### Example 5

### 2002 trial comparison between cotton plants containing the OA1 mutant Allele and DP-340 Plants

Table 1 shows data obtained from trials conducted in 2002 at nurseries in the San Joaquin Valley, Calif. comparing various characteristics between the present invention, cotton plants containing the OA1 mutant allele and commercial cotton cultivar DP-340 which does not contain the OA1 mutant allele. Five feet row length of cotton plants from each variety were picked out of a field and analyzed here in Table 1. Row three shows the plant height in centimeters, row four shows the number of FP1, row five shows the number of FP2, row six shows the number of FP3, row seven shows the number of FP4 and FP5, row eight shows the number of bolls off the main stem, row nine shows the total number of back to back bolls, row ten shows total number of bolls, row eleven shows the number of two fruiting positions at the same node on the main stem, row twelve shows the number of three or more positions at the same node on the main stem, row thirteen shows the total number of nodes on the main stem, row fourteen shows the number of indeterminate fruiting branches, row fifteen shows the number of determinate fruiting branches, row sixteen shows the number of fruiting positions on the branches, row seventeen shows the branch length to boll in centimeters, row eighteen shows the average number of total leaves per cotton plant, row nineteen shows the leaf to boll ratio, row twenty shows the number of fruiting positions off of the main stem, row twenty-one shows the seed weight in grams, row twenty-two shows the lint weight in grams and row twenty-three shows the percentage of lint.

Table 1 can be summarized as follows. Cotton plants containing the OA1 mutant allele are significantly taller than the pima variety DP-340 in that cotton plants containing the OA1 mutant allele averaged a plant height of 90.5 centimeters, while DP-340 plants averaged a plant height of 77.8 centimeters. When compared to DP-340 plants, cotton plants containing the OA1 mutant allele unexpectedly averaged 8.4 bolls off of the main stem, while DP-340 plants averaged 0.5 bolls off of the main stem. The majority of bolls produced by the OA1 plants originate off of the main stem with a few at the FP1 position, whereas, the DP-340 plants produce bolls at all positions from the main stem up to FP5. The difference in the total boll number is not statistically significant between the two types of cotton plants. OA1 plants have an average of 4.9 nodes with two fruiting positions on the main stem, while DP-340 plants have none.

DP-340 and all other commercially available/conventional cotton plants not having the OA1 mutant allele have indeterminate branches, whereas, OA1 plants have a few determinate branches which terminated at the FP1. OA1 plants have significantly fewer branches and therefore fewer fruiting positions on those branches since the branches are determinate. Subsequently, the length of an OA1 branch is shorter in length as compared with DP-340. OA1 plants take up a significantly less surface area in the field when grown side by side. In general, two to three OA1 plants will take up as much surface area as one DP-340 plant.

OA1 plants had a third fewer leaves (average of 20.6 leaves per plant) than DP-340 plants (average of 32.4 leaves per plant), so that each leaf from an OA1 plant (a single node on the main stem of OA1 plants can produce one to three fruiting positions which may result in bolls) is more efficient than the leaves of DP-340 to produce approximately the same total number of bolls on a plant.

TABLE 1
(averages per plant)

| | Cotton Plant | |
|---|---|---|
| Characteristic | OA1 | DP-340 |
| Height (cm) | 90.5 | 77.8 |
| Number of Bolls at Fruiting Position 1 (#FP1) | 1.3 | 6.6 |
| Number of Bolls at Fruiting Position 2 (#FP2) | 0.0 | 3.0 |
| Number of Bolls at Fruiting Position 3 (#FP3) | 0.0 | 0.7 |
| Number of Bolls at Fruit Positions 4/5 (#FP4) and | 0.0 | 0.1 |
| (#FP5) | | |
| Total number of bolls off of the Main Stem (B off MS) | 8.4 | 0.5 |
| Back to Back Bolls | 0.3 | 0.0 |
| Total Number of Bolls (TB) | 10.3 | 11.0 |
| Number of two fruiting positions at the same node on | 4.9 | 0.0 |
| the main stem | | |
| Number of three or more fruiting positions at the same | 0.0 | 0.0 |
| node on the main stem | | |
| Number of nodes on the main stem (#nodes) | 18.7 | 16.9 |
| Number of indeterminate fruiting branches (#FB) | 0.0 | 9.7 |
| Number of determinate fruiting branches (#FB) | 1.9 | 0.0 |
| Number of fruiting positions on the branches (#FTP) | 1.9 | 15.5 |
| Branch length to Bolls (BRB) | 81.6 | 204.9 |
| Total Number of Leaves | 20.6 | 32.4 |
| Leaf to Boll Ratio | 2/1 | 2.9/1 |
| Number of fruiting positions off of the main stem (FTP | 13.3 | 0.7 |
| off MS) | | |
| Seed Weight (g) | 20.0 | 19.5 |
| Lint Weight (g) | 12.8 | 12.5 |
| Percentage Lint (%) | 39.0 | 39.1 |

### Example 6

### 2003 Trial Comparison Between Cotton Plants Containing the OA1 Mutant Allele and DP-340 Plants

Table 2 shows data obtained from trials conducted in 2003 at nurseries in the San Joaquin Valley, Calif. comparing various characteristics between the present invention, cotton plants containing the OA1 mutant allele and commercial cotton cultivar DP-340 which does not contain the OA1 mutant allele. Five feet row length of cotton plants from each variety were picked out of a field and analyzed here in Table 1. Row three shows the plant height in centimeters, row four shows the number of FP1, row five shows the number of FP2, row six shows the number of FP3, row seven shows the number of FP4 and FP5, row eight shows the number of bolls off the main stem, row nine shows the total number of back to back bolls, row ten shows total number of bolls, row eleven shows the number of two fruiting positions at the same node on the main stem, row twelve shows the number of three or more positions at the same node on the main stem, row thirteen shows the total number of nodes on the main stem, row fourteen shows the number of indeterminate fruiting branches, row fifteen shows the number of determinate fruiting branches, row sixteen shows the number of fruiting positions on the branches, row seventeen shows the branch length to boll in centimeters, row eighteen shows the average number of total leaves per cotton plant, row nineteen shows the leaf to boll ratio, row twenty shows the number of fruiting positions off of the main stem, row twenty-one shows the seed weight in grams, row twenty-two shows the lint weight in grams and row twenty-three shows the percentage of lint.

Table 2 can be summarized as follows. Cotton plants containing the OA1 mutant allele are significantly taller than the pima variety DP-340 in that cotton plants containing the OA1 mutant allele averaged a plant height of 82.9 centimeters, while DP-340 plants averaged a plant height of 65.6 centimeters. When compared to DP-340 plants, cotton plants containing the OA1 mutant allele averaged 9.5 bolls off of the main stem, while DP-340 plants averaged 0.5 bolls off of the main stem. The majority of bolls produced by the OA1 plants originate off of the main stem with a few at the FP1 position, whereas, the DP-340 plants produce bolls at all positions from the main stem up to FP5. The difference in the total boll number is not statistically significant between the two types of cotton plants. OA1 plants have an average of 7.7 nodes with two fruiting positions on the main stem, while DP-340 plants have none.

DP-340 and all other commercially available/conventional cotton plants not having the OA1 mutant allele have indeterminate branches, whereas, OA1 plants have a few determinate branches which terminated at the FP1. OA1 plants have significantly fewer branches and therefore fewer fruiting positions on those branches since the branches are determinate. Subsequently, the length of an OA1 branch is shorter in length as compared with DP-340. OA1 plants take up a significantly less surface area in the field when grown side by side. In general, two to three OA1 plants will take up as much surface area as one DP-340 plant.

OA1 plants had a third fewer leaves (average of 19.1 leaves per plant) than DP-340 plants (average of 30.2 leaves per plant), so that each leaf from an OA1 plant (a single node on the main stem of OA1 plants can produce one to three fruiting positions which may result in bolls) is more efficient than the leaves of DP-340 to produce approximately the same total number of bolls on a plant.

TABLE 2
(averages per plant)

| | Cotton Plant | |
|---|---|---|
| Characteristic | OA1 | DP-340 |
| Height (cm) | 82.9 | 65.6 |
| Number of Bolls at Fruiting Position 1 (#FP1) | 2.6 | 7.5 |
| Number of Bolls at Fruiting Position 2 (#FP2) | 0.0 | 2.8 |
| Number of Bolls at Fruiting Position 3 (#FP3) | 0.0 | 0.9 |
| Number of Bolls at Fruit Positions 4/5 (#FP4) and | 0.0 | 0.1 |
| (#FP5) | | |
| Total number of bolls off of the Main Stem (B off MS) | 9.5 | 0.5 |
| Back to Back Bolls | 0.3 | 0.0 |
| Total Number of Bolls (TB) | 12.7 | 12.0 |
| Number of two fruiting positions at the same node on | 7.7 | 0.0 |
| the main stem | | |
| Number of three or more fruiting positions at the same | 0.8 | 0.0 |
| node on the main stem | | |
| Number of nodes on the main stem (#nodes) | 17.2 | 15.7 |
| Number of indeterminate fruiting branches (#FB) | 0.0 | 9.0 |
| Number of determinate fruiting branches (#FB) | 1.9 | 0.0 |
| Number of fruiting positions on the branches (#FTP) | 1.9 | 14.5 |
| Branch length to Bolls (BRB) | 109.7 | 218 |
| Total number of Leaves | 19.1 | 30.2 |
| Leaf to Boll Ratio | 3/2 | 5/2 |
| Number of fruiting positions on of the main stem (FTP | 20.9 | 1.7 |
| MS) | | |
| Seed Weight (g) | 22.7 | 22.2 |
| Lint Weight (g) | 14.5 | 14.4 |
| Percentage Lint (%) | 39.0 | 39.3 |

### Example 7

### 2004 Trial Comparison Between Cotton Plants Containing the OA1 Mutant Allele and DP-340 Plants

Table 3 shows data obtained from trials conducted in 2004 at nurseries in the San Joaquin Valley, Calif. comparing various characteristics between the present invention, cotton plants containing the OA1 mutant allele and commercial cotton cultivar DP-340 which does not contain the OA1 mutant allele. Five feet row length of cotton plants from each variety were picked out of a field and analyzed here in Table 1. Row three shows the plant height in centimeters, row four shows the number of FP1, row five shows the number of FP2, row six shows the number of FP3, row seven shows the number of FP4 and FP5, row eight shows the number of bolls off the main stem, row nine shows the total number of back to back bolls, row ten shows total number of bolls, row eleven shows the number of two fruiting positions at the same node on the main stem, row twelve shows the number of three or more positions at the same node on the main stem, row thirteen shows the total number of nodes on the main stem, row fourteen shows the number of indeterminate fruiting branches, row fifteen shows the number of determinate fruiting branches, row sixteen shows the number of fruiting positions on the branches, row seventeen shows the branch length to boll in centimeters, row eighteen shows the average number of total leaves per cotton plant, row nineteen shows the leaf to boll ratio, row twenty shows the number of fruiting positions off of the main stem, row twenty-one shows the seed weight in grams, row twenty-two shows the lint weight in grams and row twenty-three shows the percentage of lint.

Table 3 can be summarized as follows. Cotton plants containing the OA1 mutant allele are significantly taller than the pima variety DP-340 in that cotton plants containing the OA1 mutant allele averaged a plant height of 79.4 centimeters, while DP-340 plants averaged a plant height of 65.6 centimeters. When compared to DP-340 plants, cotton plants containing the OA1 mutant allele averaged 10.3 bolls off of the main stem, while DP-340 plants averaged no bolls off of the main stem. The majority of bolls produced by the OA1 plants originate off of the main stem with a few at the FP1 position, whereas, the DP-340 plants produce bolls at all positions from the main stem up to FP5. The difference in the total boll number is statistically significant between the two types of cotton plants. OA1 plants have an average of 6.2 nodes with two fruiting positions on the main stem, while DP-340 plants have none.

OA1 plants had a third fewer leaves (average of 21.3 leaves per plant) than DP-340 plants (average of 32.6 leaves per plant), so that each leaf from an OA1 plant (a single node on the main stem of OA1 plants can produce one to three fruiting positions which may result in bolls) is more efficient than the leaves of DP-340 to produce approximately the same total number of bolls on a plant.

TABLE 3
2004 South Site (Averages per plant)

| | Cotton Plant | |
|---|---|---|
| Characteristic | OA1 | DP-340 |
| Height (cm) | 79.4 | 69.5 |
| Number of Bolls at Fruiting Position 1 (#FP1) | 4.5 | 7.7 |
| Number of Bolls at Fruiting Position 2 (#FP2) | 0.0 | 2.6 |
| Number of Bolls at Fruiting Position 3 (#FP3) | 0.0 | 0.7 |
| Number of Bolls at Fruit Positions 4/5 (#FP4) and | 0.0 | 0.0 |
| (#FP5) | | |
| Total number of bolls off of the Main Stem (B off MS) | 10.3 | 0.0 |
| Back to Back Bolls | 0.4 | 0.0 |
| Total Number of Bolls (TB) | 15.1 | 11.5 |
| Number of two fruiting positions at the same node on | 6.2 | 0.0 |
| the main stem | | |
| Number of three or more fruiting positions at the same | 0.1 | 0.0 |
| node on the main stem | | |
| Number of nodes on the main stem (#nodes) | 18.3 | 16.6 |
| Number of indeterminate fruiting branches (#FB) | 0.0 | 10.0 |
| Number of determinate fruiting branches (#FB) | 3.0 | 0.0 |
| Number of fruiting positions on the branches (#FTP) | 3 | 16 |
| Branch length to Bolls (BRB) | 141.1 | 226.6 |
| Total number of Leaves | 21.3 | 32.6 |
| Leaf to Boll Ratio | 1.41/1 | 2.83/1 |
| Number of fruiting positions on of the main stem (FTP | 15.6 | 0.5 |
| MS) | | |
| Seed Weight (g) | 22.8 | 18.6 |
| Lint Weight (g) | 16.3 | 13.3 |
| Percentage Line (%) | 41.7 | 41.7 |

### Example 8

### 2004 Trial Comparison Between Cotton Plants Containing the OA1 Mutant Allele and DP-340 Plants

Table 4 shows data obtained from trials conducted in 2004 at nurseries in the San Joaquin Valley, Calif. comparing various characteristics between the present invention, cotton plants containing the OA1 mutant allele and commercial cotton cultivar DP-340 which does not contain the OA1 mutant allele. Five feet row length of cotton plants from each variety were picked out of a field and analyzed here in Table 1. Row three shows the plant height in centimeters, row four shows the number of FP1, row five shows the number of FP2, row six shows the number of FP3, row seven shows the number of FP4 and FP5, row eight shows the number of bolls off the main stem, row nine shows the total number of back to back bolls, row ten shows total number of bolls, row eleven shows the number of two fruiting positions at the same node on the main stem, row twelve shows the number of three or more positions at the same node on the main stem, row thirteen shows the total number of nodes on the main stem, row fourteen shows the number of indeterminate fruiting branches, row fifteen shows the number of determinate fruiting branches, row sixteen shows the number of fruiting positions on the branches, row seventeen shows the branch length to boll in centimeters, row eighteen shows the average number of total leaves per cotton plant, row nineteen shows the leaf to boll ratio, row twenty shows the number of fruiting positions off of the main stem, row twenty-one shows the seed weight in grams, row twenty-two shows the lint weight in grams and row twenty-three shows the percentage of lint.

Table 4 can be summarized as follows. Cotton plants containing the OA1 mutant allele are significantly taller than the pima variety DP-340 in that cotton plants containing the OA1 mutant allele averaged a plant height of 89.2 centimeters, while DP-340 plants averaged a plant height of 96.2 centimeters. When compared to DP-340 plants, cotton plants containing the OA1 mutant allele averaged 11.1 bolls off of the main stem, while DP-340 plants averaged no bolls off of the main stem. The majority of bolls produced by the OA1 plants originate off of the main stem with a few at the FP1 position, whereas, the DP-340 plants produce bolls at all positions from the main stem up to FP5. The difference in the total boll number is statistically significant between the two types of cotton plants. OA1 plants have an average of 6.6 nodes with two fruiting positions on the main stem, while DP-340 plants have none.

OA1 plants had a third fewer leaves (average of 21.7 leaves per plant) than DP-340 plants (average of 34.2 leaves per plant), so that each leaf from an OA1 plant (a single node on the main stem of OA1 plants can produce one to three fruiting positions which may result in bolls) is more efficient than the leaves of DP-340 to produce approximately the same total number of bolls on a plant.

TABLE 4
North Site (averages per plant)

| | Cotton Culitvar | |
|---|---|---|
| Characteristic | OA1 | DP-340 |
| Height (cm) | 89.2 | 96.2 |
| Number of Bolls at Fruiting Position 1 (#FP1) | 3.8 | 7.7 |
| Number of Bolls at Fruiting Position 2 (#FP2) | 0.0 | 2.0 |
| Number of Bolls at Fruiting Position 3 (#FP3) | 0.0 | 0.3 |
| Number of Bolls at Fruit Positions 4/5 (#FP4) and | 0.0 | 0.0 |
| (#FP5) | | |
| Total number of bolls off of the Main Stem (B off MS) | 11.1 | 0.0 |
| Back to Back Bolls | 0.3 | 0.0 |
| Total Number of Bolls (TB) | 15.5 | 10.3 |
| Number of two fruiting positions at the same node on | 6.6 | 0.0 |
| the main stem | | |
| Number of three or more fruiting positions at the same | 0.2 | 0.0 |
| node on the main stem | | |
| Number of nodes on the main stem (#nodes) | 18.7 | 18.9 |
| Number of indeterminate fruiting branches (#FB) | 0.0 | 10.3 |
| Number of determinate fruiting branches (#FB) | 2.8 | 0.0 |
| Number of fruiting positions on the branches (#FTP) | 3.0 | 15.3 |
| Branch length to Bolls (BRB) | 149.7 | 233.1 |
| Total # of Leaves | 21.7 | 34.2 |
| Leaf to Boll Ratio | 1.4/1 | 3.3/1 |
| Number of fruiting positions on of the main stem (FTP | 17.2 | 2.5 |
| MS) | | |
| Seed Weight (g) | 22.7 | 16.7 |
| Lint Weight (g) | 15.7 | 11.2 |
| Percentage Lint (%) | 40.9 | 40.1 |

### Example 9

### 2005 Trial Comparison Between Cotton Plants Containing the OA1 Mutant Allele and DP-340 Plants

Table 5 shows data obtained from trials conducted in 2005 at nurseries in the San Joaquin Valley, Calif. comparing various characteristics between the present invention, cotton plants containing the OA1 mutant allele and commercial cotton cultivar DP-340 which does not contain the OA1 mutant allele. Five feet row length of cotton plants from each variety were picked out of a field and analyzed here in Table 1. Row three shows the plant height in centimeters, row four shows the number of FP1, row five shows the number of FP2, row six shows the number of FP3, row seven shows the number of FP4 and FP5, row eight shows the number of bolls off the main stem, row nine shows the total number of back to back bolls, row ten shows total number of bolls, row eleven shows the number of two fruiting positions at the same node on the main stem, row twelve shows the number of three or more positions at the same node on the main stem, row thirteen shows the total number of nodes on the main stem, row fourteen shows the number of indeterminate fruiting branches, row fifteen shows the number of determinate fruiting branches, row sixteen shows the number of fruiting positions on the branches, row seventeen shows the branch length to boll in centimeters, row eighteen shows the average number of total leaves per cotton plant, row nineteen shows the leaf to boll ratio, row twenty shows the number of fruiting positions off of the main stem, row twenty-one shows the seed weight in grams, row twenty-two shows the lint weight in grams and row twenty-three shows the percentage of lint.

Table 5 can be summarized as follows. Cotton plants containing the OA1 mutant allele are taller than the pima variety DP-340 in that cotton plants containing the OA1 mutant allele averaged a plant height of 67.4 centimeters, while DP-340 plants averaged a plant height of 62.8 centimeters. When compared to DP-340 plants, cotton plants containing the OA1 mutant allele averaged 9.6 bolls off of the main stem, while DP-340 plants averaged 0.3 bolls off of the main stem. The majority of bolls produced by the OA1 plants originate off of the main stem with a few at the FP1 position, whereas, the DP-340 plants produce bolls at all positions from the main stem up to FP5. The difference in the total boll number is not statistically significant between the two types of cotton plants. OA1 plants have an average of 4.3 nodes with two fruiting positions on the main stem, while DP-340 plants have none.

OA1 plants had a third fewer leaves (average of 13.7 leaves per plant) than DP-340 plants (average of 31.9 leaves per plant), so that each leaf from an OA1 plant (a single node on the main stem of OA1 plants can produce one to three fruiting positions which may result in bolls) is more efficient than the leaves of DP-340 to produce approximately the same total number of bolls on a plant.

TABLE 5

| | Cotton Plant | |
|---|---|---|
| Characteristic | OA-1 | DP-340 |
| Height (cm) | 67.4 | 62.8 |
| Number of Bolls at Fruiting Position 1 (#FP1) | 2.0 | 6.4 |
| Number of Bolls at Fruiting Position 2 (#FP2) | 0.0 | 3.4 |
| Number of Bolls at Fruiting Position 3 (#FP3) | 0.0 | 1.1 |
| Number of Bolls at Fruit Positions 4/5 (#FP4) and | 0.0 | 0.2 |
| (#FP5) | | |
| Total number of bolls off of the Main Stem (B off MS) | 9.6 | 0.3 |
| Back to Back Bolls | 0.5 | 0.0 |
| Total Number of Bolls (TB) | 11.9 | 12.3 |
| Number of two fruiting positions at the same node on | 4.3 | 0.0 |
| the main stem | | |
| Number of three or more fruiting positions at the same | 0.0 | 0.0 |
| node on the main stem | | |
| Number of nodes on the main stem (#nodes) | 11.7 | 16.1 |
| Number of indeterminate fruiting branches (#FB) | 0.0 | 9.4 |
| Number of determinate fruiting branches (#FB) | 2.0 | 0.0 |
| Number of fruiting positions on the branches (#FTP) | 2.0 | 15.8 |
| Branch length to Bolls (BRB) | 82.0 | 244.7 |
| Total number of Leaves | 13.7 | 31.9 |
| Leaf to Boll Ratio | 1.15/1 | 2.59/1 |
| Number of fruiting positions on of the main stem (FTP | 12.3 | 0.6 |
| MS) | | |
| Seed Weight (g) | 17.1 | 16.7 |
| Lint Weight (g) | 10.9 | 11.7 |
| Percentage Lint (%) | 38.9 | 41.2 |

### Example 10

### Summary of Tables 1-5 Comparing Cotton Plants Containing the OA1 mutant allele with DP-340 Plants

Table 6 shows data obtained from trials conducted in 2003-2005 in nurseries in California comparing various characteristics between the present invention, cotton plants containing the OA1 mutant allele, and commercial cotton cultivar DP-340 which does not contain the OA1 mutant allele. Five feet row length of cotton plants from each variety were picked out of a field and analyzed here in Table 1. Row three shows the plant height in centimeters, row four shows the number of FP1, row five shows the number of FP2, row six shows the number of FP3, row seven shows the number of FP4 and FP5, row eight shows the number of bolls off the main stem, row nine shows the total number of back to back bolls, row ten shows total number of bolls, row eleven shows the number of two fruiting positions at the same node on the main stem, row twelve shows the number of three or more positions at the same node on the main stem, row thirteen shows the total number of nodes on the main stem, row fourteen shows the number of indeterminate fruiting branches, row fifteen shows the number of determinate fruiting branches, row sixteen shows the number of fruiting positions on the branches, row seventeen shows the branch length to boll in centimeters, row eighteen shows the average number of total leaves per cotton plant, row nineteen shows the leaf to boll ratio, row twenty shows the number of fruiting positions off of the main stem, row twenty-one shows the seed weight in grams, row twenty-two shows the lint weight in grams and row twenty-three shows the percentage of lint.

Table 6 can be summarized as follows. Cotton plants containing the OA1 mutant allele are taller than the pima variety DP-340 in that cotton plants containing the OA1 mutant allele averaged a taller plant height of 81.2 centimeters, while DP-340 plants averaged a plant height of 74.2 centimeters. When compared to DP-340 plants, cotton plants containing the OA1 mutant allele unexpectedly averaged 9.8 bolls off of the main stem, while DP-340 plants averaged 0.2 bolls off of the main stem. The majority of bolls produced by the OA1 plants originate off of the main stem with a few at the FP1 position, whereas, the DP-340 plants produce bolls at all positions from the main stem up to FP5. The difference in the total boll number is statistically significant between the two types of cotton plants. OA1 plants have an average of 6.2 nodes with two fruiting positions on the main stem, while DP-340 plants have none.

OA1 plants had a third fewer leaves (average of 19.0 leaves per plant) than DP-340 plants (average of 32.1 leaves per plant), so that each leaf from an OA1 plant (a single node on the main stem of OA1 plants can produce one to three fruiting positions which may result in bolls) is more efficient than the leaves of DP-340 to produce approximately the same total number of bolls on a plant.

TABLE 6
(averages per plant)

| | Cotton Plant | |
|---|---|---|
| Characteristic | OA-1 | DP-340 |
| Height (cm) | 81.2 | 74.2 |
| Number of Bolls at Fruiting Position 1 (#FP1) | 2.9 | 7.8 |
| Number of Bolls at Fruiting Position 2 (#FP2) | 0.0 | 2.2 |
| Number of Bolls at Fruiting Position 3 (#FP3) | 0.0 | 0.7 |
| Number of Bolls at Fruit Positions 4/5 (#FP4) and | 0.0 | 0.1 |
| (#FP5) | | |
| Total number of bolls off of the Main Stem (B off MS) | 9.8 | 0.2 |
| Back to Back Bolls | 0.3 | 0.0 |
| Total Number of Bolls (TB) | 13.2 | 11.5 |
| Number of two fruiting positions at the same node on | 6.2 | 0.0 |
| the main stem | | |
| Number of three or more fruiting positions at the same | 0.3 | 0.0 |
| node on the main stem | | |
| Number of nodes on the main stem (#nodes) | 16.7 | 16.8 |
| Number of indeterminate fruiting branches (#FB) | 0.0 | 9.7 |
| Number of determinate fruiting branches (#FB) | 2.3 | 0.0 |
| Number of fruiting positions on the branches (#FTP) | 2.3 | 15.3 |
| Branch length to Bolls (BRB) | 135.1 | 233.0 |
| Total number of Leaves | 19.0 | 32.1 |
| Leaf to Boll Ratio | 1.44/1 | 2.8/1 |
| Number of fruiting positions off of the main stem (FTP | 16.5 | 1.3 |
| MS) | | |
| Seed Weight (g) | 21.2 | 19.0 |
| Lint Weight (g) | 14.1 | 12.8 |
| Percentage Lint (%) | 39.9 | 40.2 |

### Example 11

### Comparison of Fiber Properties Among G. barbadense Varieties Containing The OA1 Mutant Allele with Phytogen 800 and DP-340

Table 7 compares the fiber qualities among G. barbadense cotton varieties containing the OA1 mutant allele, pima quality G. barbadense cotton varieties containing the OA1 mutant allele with commercial cotton pima varieties Phytogen 800 and DP-340 which do not contain the OA1 mutant allele. The data for Pima cotton varieties containing the OA1 mutant allele were collected from nine cotton lines containing the OA1 mutant allele. Each variety's figures are an average taken from five replications with three fiber readings per replication. Trials were conducted in 2006 in the San Joaquin Valley, Calif. The type of cotton varieties are listed in column one, column two shows the micronaire, column three shows the length in inches, column four shows the uniformity ratio, column five shows the fiber strength, column six shows the fiber elongation, column seven shows the maturity rating, column eight shows the SCI, column nine shows the SFI, column ten shows the CSP, column eleven shows the CG, column twelve shows the B, column thirteen shows the RD, column fourteen shows the area, column fifteen shows the LF and column sixteen shows the CLLF. This table shows the vast array of fiber qualities and fiber color in new cotton varieties with the mutant allele.

TABLE 7

| | Fiber Characteristic | | | | | | | | | | | | | | |
|---|---|---|---|---|---|---|---|---|---|---|---|---|---|---|---|
| Cotton Plant | MIC | LEN | UR | T1 | E1 | MR | SCI | SFI | CSP | CG | B | RD | Area | LF | CLLF |
| Pima quality, | | | | | | | | | | | | | | | |
| OA1 varieties | | | | | | | | | | | | | | | |
| Exp.-45 | 3.40 | 1.46 | 90.20 | 49.10 | 6.90 | 88 | 112 | 3.20 | 2876 | 1 | 8.10 | 78.30 | 0.10 | 1 | 1 |
| Exp.-52 | 3.90 | 1.50 | 93.60 | 59.20 | 6.50 | 90 | 125 | 2.10 | 2977 | 1 | 7.70 | 78.30 | 0.70 | 7 | 5 |
| Exp.-80 | 3.10 | 1.49 | 91.30 | 63.20 | 3.70 | 90 | 122 | 3.40 | 3000 | 1 | 9.10 | 79.50 | 0.20 | 2 | 2 |
| Exp.-32 | 3.30 | 1.52 | 92.20 | 53.10 | 5.90 | 88 | 126 | 2.90 | 2934 | 1 | 9.10 | 79.10 | 1.10 | 11 | 7 |
| Exp.-21 | 3.20 | 1.49 | 92.70 | 57.10 | 7.00 | 88 | 125 | 2.40 | 2889 | 22-1 | 10.30 | 76.00 | 0.90 | 9 | 6 |
| Exp.-130 | 3.60 | 1.48 | 91.90 | 49.30 | 6.40 | 89 | 119 | 1.70 | | 21-2 | 8.90 | 78.10 | 0.00 | 0 | 1 |
| Exp.-69 | 4.50 | 1.40 | 89.00 | 46.60 | 7.00 | 90 | 89 | 3.00 | 2496 | 2 | 11.20 | 72.20 | | | |
| Exp.-23 | 5.10 | 1.43 | 89.00 | 44.60 | 6.70 | 92 | 84 | 2.50 | 2313 | 2 | 15.60 | 71.10 | | | |
| Exp.-87 | 4.60 | 1.43 | 90.20 | 43.40 | 7.40 | 90 | 97 | 2.70 | 2584 | 1 | 10.00 | 75.00 | | | |
| Conventional | | | | | | | | | | | | | | | |
| varieties | | | | | | | | | | | | | | | |
| Phytogen 800 | 4.43 | 1.42 | 88.68 | 45.03 | 8.91 | 88.73 | 87 | 3.54 | 2447 | | 10.67 | 69.73 | 0.67 | 7 | 3 |
| DP 340 | 4.56 | 1.41 | 88.92 | 44.28 | 8.20 | 89.56 | 87 | 3.21 | 2466 | 1.6 | 10.84 | 70.92 | 0.25 | 3 | 2 |

### Example 11

### Comparison of Fiber Properties Among Some G. hirsutum Varieties Containing the OA1 Mutant Allele

Table 8 compares the fiber properties among some G. hirsutum varieties containing the OA1 mutant allele. The data for upland/acala cotton varieties containing the OA1 mutant allele were collected from six cotton lines containing the OA1 mutant allele. Each variety's figures are an average taken from five replications with three fiber readings per replication. Trials were conducted in 2000 in the San Joaquin Valley, Calif. The type of cotton plants are listed in column one, column two shows the micronaire, column three shows the length in inches, column four shows the uniformity ratio, column five shows the fiber strength, column six shows the fiber elongation. This shows the diversity of fiber qualities within the G. hirsutum varieties containing the OA1 mutant allele.

TABLE 8

| Variety | Micronaire | Length | Uniformity | T1 |
|---|---|---|---|---|
| Exp.-224 | 5.10 | 1.12 | 78.23 | 31.06 |
| Exp.-203 | 5.21 | 1.13 | 78.54 | 32.01 |
| Exp.-274 | 5.07 | 1.18 | 79.85 | 32.17 |
| Exp.-255 | 4.88 | 1.22 | 81.90 | 32.66 |
| Exp.-315 | 4.35 | 1.30 | 85.73 | 35.96 |
| Exp.-356 | 4.22 | 1.29 | 85.56 | 36.37 |

### Example 12

### Comparison of Vascular Staining Among G. barbadense and G. hirsutum Varieties Containing the OA1 Mutant Allele in a Fusarium Race-4 Infested Field

Table 9 compares the vascular staining among some G. barbadense and G. hirsutum varieties containing the OA1 mutant allele in a Fusarium Race-4 field. This test was conducted in 2007 at an OA nursery site to determine the susceptibility of each variety to Fusarium Race-4. The left column is comprised of some experimental varieties that have the OA1 mutant allele. The middle column indicates whether or not the varieties had a vascular stain within the base of the main stem or not. The stain is around the base of the main stem and can be found by striping the main stem in half. No Stain reading is preferred because a stain shows susceptibility to the disease. The right column shows the yield and performance of that particular variety. A very good rating is the best and very poor is the worst rating. The important point being that we developed very diverse cultivars that have the OA1 mutant allele

TABLE 9

| Variety | Stained vs. Not Stain | Yield/Performance |
|---|---|---|
| Exp.-113 | No Stain | Very Good Yield |
| Exp.-139 | No Stain | Good Yield |
| Exp.-72 | No Stain | Ok Yield |
| Exp.-166 | No Stain | Poor Yield |
| Exp.-681 | Stained | Very Poor Yield |

### Example 13

### Comparison of Dead Plants Among G. barbadense and G. hirsutum Varieties Containing the OA1 Mutant Allele in a Fusarium Race-4 Field

Table 10 compares the percentage of dead plants among G. barbadense and G. hirsutum varieties containing the OA1 mutant allele in a Fusarium Race-4 field. This test was conducted in 2007 at an OA nursery site to determine approximately how many plants will die in a field infested with Fusarium Race-4. The far left column shows OA1 mutant allele varieties with three commercial varieties as checks. The second column shows the stand, which is a rating of how well the plant spacing is, from one to five with five being preferred. Each of the next five columns represents a date the field was evaluated. The percentage shows how many new dead plants there are since the last reading was taken. So there is a compounding effect that will happen when adding up the total, which is the last column. This last column tells the percentage of plants that died throughout the season. The more susceptible the variety, the more plants that die. The lowest percentage is preferred as the variety is more tolerant or resistant. This again shows the range of Race-4 resistance in the variety of cultivars that have the OA1 mutant allele.

TABLE 10

| Variety | Stand | May 16, 2007 | Jun. 7, 2007 | Jun. 20, 2007 | Jul. 2, 2007 | Jul. 30, 2007 | Total % dead |
|---|---|---|---|---|---|---|---|
| Ex-319 | 4 | 0% | 0% | 0% | 0% | 0% | 0% |
| Ex-339 | 5 | 0% | 0% | 0% | 0% | 0% | 0% |
| Ex-35 | 5 | 10% | 25% | 5% | 0% | 0% | 36% |
| Phy-800 | 5 | 20% | 25% | 5% | 0% | 0% | 43% |
| Ex-163 | 5 | 0% | 40% | 10% | 0% | 0% | 46% |
| Ex-398 | 5 | 0% | 80% | 10% | 5% | 0% | 83% |
| DP-340 | 4 | 5% | 50% | 70% | 25% | 10% | 92% |
| DP-744 | 5 | 70% | 90% | 95% | 1 plant | 1 plant | 99% |
| Ex-39 | 4 | 5% | 100% | 100% | 100% | 100% | 100% |
| Ex-309 | 5 | 0% | 100% | 100% | 100% | 100% | 100% |

### Example 14

### Comparison of Seed Appearance after Ginning Among G. barbadense and G. hirsutum Varieties Containing the OA1 Mutant Allele.

Table 11 compares the seed appearance after ginning among G. barbadense and G. hirsutum varieties containing the OA1 mutant allele. The left column indicates the variety, all of which have the OA1 mutant allele. The right column indicates what the seed looks like after ginning. If the seed is completely covered by linters, then the seed is termed “Fuzzy.” If the seed has a very small amount of linters protruding from the seed, then the seed is “Very Naked.” This table ranges from the fuzziest seed (top) to the most naked seed (bottom). Again showing the range of varieties that has the OA1 mutant allele.

TABLE 11

| Variety | Seed appearance after ginning |
|---|---|
| MSL-4 | Fuzzy |
| Exp.-142 | Fuzzy |
| Exp.-86 | Fuzzy |
| Exp.-107 | Fuzzy |
| HQ 23-41 | Fuzzy |
| 12-18 | Semi-naked |
| 54-1-2 | Semi-naked |
| MSL-C | Semi-naked |
| HQ 23-112 | Naked |
| HQ 59 Series | Very Naked |
| HQ 7 Series | Very Naked |

### Deposit Information

A deposit of the proprietary cotton seeds containing the OA1 mutant allele of this invention is maintained by O & A Enterprises, Inc., P.O. Box 1440, Maricopa, Ariz. 85239. Access to this deposit will be available during the pendency of this application to persons determined by the Commissioner of Patents and Trademarks to be entitled thereto under 37 CFR §1.14 and 35 USC §122.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A cotton seed containing a mutant allele designated OA1, wherein a representative sample of seed containing said allele has been deposited under ATCC Accession No. PTA-______ and/or at O&A Enterprises Incorporated.

2. The allele of claim 1, wherein said allele is a dominant or a partially dominant mutant allele.

3. A cotton plant, or a part thereof, produced by growing the seed of claim 1.

4. A tissue culture of cells produced from the plant of claim 3, wherein said cells of the tissue culture are produced from a plant part selected from the group consisting of leaves, pollen, embryos, cotyledons, hypocotyl, meristematic cells, roots, root tips, pistils, anthers, flowers, and stems.

5. A protoplast produced from the plant of claim 3.

6. A protoplast produced from the tissue culture of claim 4.

7. A cotton plant regenerated from said tissue culture of claim 3, wherein said plant contains said mutant allele OA1.

8. A method for producing hybrid cotton seed comprising crossing a first parent cotton plant with a second parent cotton plant and harvesting the resultant hybrid cotton seed, wherein said first or second parent cotton plant is the cotton plant of claim 3.

9. A cotton plant, or a regenerable part thereof, produced by growing the cotton seed of claim 8.

10. A cotton seed or plant derived from seeds deposited under ATCC Accession No. PTA-______ and/or at O&A Enterprises Incorporated wherein said seed or plant contains an allele designated OA1 for the Main Stem Load structure on a cotton plant.

11. The method of claim 8, wherein said first or said second parent cotton plant is a G. hirsutum cotton plant.

12. The method of claim 8, wherein said first or said second parent cotton plant is a G. barbadense cotton plant.

13. The cotton plant produced by the method of claim 11.

14. The cotton plant produced by the method of claim 12.

15. A cotton plant having three or less branches produced by the method of claim 11.

16. A cotton plant having three or less branches produced by the method of claim 12.

17. The cotton plant of claim 3, wherein said plant has no branches.

18. The cotton plant of claim 3, wherein said plant has an average of no more than three determinate branches.

19. The cotton plant of claim 3, wherein said plant has at least five bolls off of the main stem.

20. The cotton plant of claim 3, wherein said cotton plant at maturity has significantly less leaves as compared to a conventional plant.

21. The cotton plant of claim 3, wherein said cotton plant has less than five bolls off of the FP1 part of any branch.

22. The cotton plant of claim 3, wherein said cotton plant has a significantly reduced plant width when compared to a conventional plant.

23. The cotton plant of claim 3, wherein said cotton plant has significantly less branch length to bolls as compared to a conventional plant.

24. The cotton plant of claim 3, wherein said cotton plant has a reduced number of leaves on average per plant when compared to conventional plant.

25. The cotton plant of claim 3, wherein said cotton plant has a reduced number of branches on average per plant when compared to conventional plant.

26. The cotton plant of claim 3, wherein said cotton plant has a lower leaf to boll ratio on average per plant when compared to conventional plant.

27. The cotton plant of claim 3, wherein said cotton plant averages more than one fruiting position per fruiting node on average per plant.

28. The cotton plant of claim 3, wherein said cotton plants have a high tolerance or resistance to Fusarium Race-4 wilt.
